(19) United States
(12) Patent Application Publication
(10) Pub. No.: US 2022/0125789 A1
(43) Pub. Date: Apr. 28, 2022

(54) PHARMACEUTICAL PREPARATION OF FRUQUINTINIB AND USE THEREOF

(71) Applicant: Hutchison Medipharma Limited, Shanghai (CN)

(72) Inventors: Zhongzhou Liu, Shanghai (CN); Jinli Wu, Shanghai (CN); Chongdong Fu, Shanghai (CN)

(73) Assignee: Hutchison Medipharma Limited, Shanghai (CN)

(21) Appl. No.: 17/293,790

(22) PCT Filed: Nov. 15, 2019
(86) PCT No.: PCT/CN2019/118881

§ 371 Date: May 13, 2021

(30) Foreign Application Priority Data
Nov. 15, 2018 (CN) 201811358991.5

(51) Int. Cl. A61K 31/517; A61K 31/337; A61K 31/5377; A61K 9/48

(52) U.S. Cl. CPC A61K 31/517; A61K 9/4866; A61K 31/5377; A61K 31/337

(57) ABSTRACT

A pharmaceutical composition of fruquintinib comprising filler and its preparing process are disclosed. The filler is selected from starch, microcrystalline cellulose or a combination thereof. The composition is in the form of a tablet or capsule and can be used in the treatment of cancer, such as colorectal cancer, non-small cell lung cancer, and gastric cancer.

### TECHNICAL FIELD

The present invention relates to the field of pharmaceutical preparations, in particular to a pharmaceutical preparation of fruquintinib and use thereof.

### BACKGROUND

Fruquintinib, a compound of Formula A having the chemical name 6-((6,7-dimethoxyquinazolin-4-yl)oxy)-N,2-dimethyl-benzofuran-3-carboxamide, is a novel high potent VEGFR selective inhibitor mainly used for the treatment of cancers such as colorectal cancer, non-small cell lung cancer and gastric cancer.

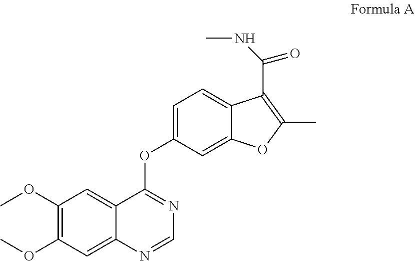

Chinese patent CN101575333B (SU Weiguo, et al.) disclosed the synthesis of fruquintinib and its use in the treatment of tumors, age-related macular degeneration or chronic inflammation.

Chinese Patent Application No. 201410456350.9 (WU Zhenping, et al.) disclosed the crystal forms and solvates of fruquintinib.

Clinical studies have shown that fruquintinib has good tolerance and significant anti-tumor activity in patients with cancer, and its clinical dose is 4 mg once a day (p.o.) or 5 mg once a day (p.o.) for 3 weeks, followed by 1 week interval.

The fruquintinib bulk drug substance has a long fibrous appearance and is adhesive. Therefore, when the pharmaceutical composition is prepared by a conventional method, it is easy to block the mesh hole during sieving, which causes difficulty in processing the formulation and affects the uniformity of drug content.

In view of the excellent activity of fruquintinib, there is an urgent need for an orally administered fruquintinib composition which has good bioavailability and is easy to prepare.

It has now been found that by appropriately selecting the pharmaceutical auxiliary materials and/or controlling the particle size of fruquintinib in the composition, a pharmaceutical composition comprising fruquintinib having excellent processing properties can be obtained, and the oral preparation prepared from such pharmaceutical composition has a high dissolution rate of fruquintinib and good bioavailability.

### SUMMARY OF THE INVENTION

The present invention provides a pharmaceutical composition comprising fruquintinib, in particular an oral pharmaceutical composition comprising the active ingredient fruquintinib and fillers and optionally a lubricant.

In one embodiment of the invention, the filler is selected from the group consisting of starch, microcrystalline cellulose and a combination thereof. In a particular embodiment of the invention, the filler is starch. In another particular embodiment of the invention, the filler is microcrystalline cellulose. In some embodiments of the invention, the filler is a combination of starch and microcrystalline cellulose, wherein the weight ratio of microcrystalline cellulose to starch is (0.9-1.1):1. In other embodiments of the invention, the filler is a combination of starch and microcrystalline cellulose, wherein the weight ratio of microcrystalline cellulose to starch is about 1:1.

In a particular embodiment of the invention, the lubricant is selected from the group consisting of magnesium stearate, talc and a combination thereof. In a particular embodiment of the invention, the lubricant is talc. In another particular embodiment of the invention, the lubricant is magnesium stearate.

In a particular embodiment of the invention, the weight ratio of the lubricant to the filler is from about 1:50 to 1:1000, such as from about 1:100 to 1:500.

In a particular embodiment of the invention, no lubricant is used.

In some embodiments of the present invention, in order to ensure that the prepared pharmaceutical composition meets the dissolution requirements of the ordinary immediate release solid preparation, the fruquintinib bulk drug substance is subjected to micronized pulverization treatment, and the particle size range (D90) is controlled to be less than about 35 μm, for example, the particle size range (D90) is controlled in the range of about 5˜30 μm.

In some embodiments of the invention, the particle size range (D90) of the fruquintinib bulk drug substance is controlled to be less than about 15 μm, such as to control the particle size range (D90) to be in the range of about 5˜15 μm. In a particular embodiment of the invention, the fruquintinib bulk drug substance has a particle size (D90) of about 7.9 μm. In a particular embodiment of the invention, the fruquintinib bulk drug substance has a particle size (D90) of about 10.4 μm.

In other embodiments of the invention, the fruquintinib bulk drug substance has a particle size (D90) of about 18.5 μm.

In another embodiment of the invention, the fruquintinib bulk drug substance has a particle size (D90) of about 35 μm.

In the pharmaceutical composition of the present invention, the content of the active ingredient fruquintinib is in the range of about 0.001 wt % to 5 wt %, such as 0.01 to 5% by weight, such as 0.05 wt %, 1 wt %, 2 wt %, 3 wt %, and the like, based on the total weight of the pharmaceutical composition.

In one embodiment of the invention, the invention provides a pharmaceutical composition comprising fruquintinib, which comprises the following components in parts by weight:

| | | |
|---|---|---|
| Fruquintinib | 1 | part |
| Filler | 30-1200 | parts |
| Lubricant | 0-12 | parts. |

In a particular embodiment of the invention, the pharmaceutical composition comprises the following components in parts by weight:

| | | |
|---|---|---|
| Fruquintinib | 1 | part |
| Filler | 30-100 | parts |
| Lubricant | 0-12 | parts. |

In a particular embodiment of the invention, the pharmaceutical composition comprises the following components in parts by weight:

| | | |
|---|---|---|
| Fruquintinib | 1 | part |
| Filler | 1000-1200 | parts |
| Lubricant | 0-1 | part. |

In a particular embodiment of the invention, the pharmaceutical composition comprises the following components in parts by weight:

| | | |
|---|---|---|
| Fruquintinib | 1 | part |
| Starch | 1040 | parts. |

In a particular embodiment of the invention, the pharmaceutical composition comprises the following components in parts by weight:

| | | |
|---|---|---|
| Fruquintinib | 1 | part |
| Microcrystalline cellulose | 52 | parts. |

In a particular embodiment of the invention, the pharmaceutical composition comprises the following components in parts by weight:

| | | |
|---|---|---|
| Fruquintinib | 1 | part |
| Starch | 20-520 | parts |
| Microcrystalline cellulose | 20-520 | parts |
| Talc | 0.1-12 | parts. |

In a particular embodiment of the invention, the pharmaceutical composition comprises the following components in parts by weight:

| | | |
|---|---|---|
| Fruquintinib | 1 | part |
| Starch | 20-520 | parts |
| Microcrystalline cellulose | 20-520 | parts |
| Magnesium stearate | 0.1-12 | parts. |

In a particular embodiment of the invention, the pharmaceutical composition comprises the following components in parts by weight:

| | | |
|---|---|---|
| Fruquintinib | 1 | part |
| Starch | 500-520 | parts |
| Microcrystalline cellulose | 500-520 | parts |
| Talc | 2-12 | parts. |

In a particular embodiment of the invention, the pharmaceutical composition comprises the following components in parts by weight:

| | | |
|---|---|---|
| Fruquintinib | 1 | part |
| Starch | 500-520 | parts |
| Microcrystalline cellulose | 500-520 | parts |
| Magnesium stearate | 2-12 | parts. |

In a particular embodiment of the invention, the pharmaceutical composition comprises the following components in parts by weight:

| | | |
|---|---|---|
| Fruquintinib | 1 | part |
| Starch | 20-30 | parts |
| Microcrystalline cellulose | 20-30 | parts |
| Magnesium stearate | 0.1-1 | part. |

In a particular embodiment of the invention, the pharmaceutical composition comprises the following components in parts by weight:

| | | |
|---|---|---|
| Fruquintinib | 1 | part |
| Starch | 20-30 | parts |
| Microcrystalline cellulose | 20-30 | parts |
| Talc | 0.1-1 | part. |

The above pharmaceutical compositions provided by the invention have the advantages of good powder flowability and mixing uniformity and can meet the needs of large scale capsule production.

The above pharmaceutical compositions provided by the present invention, wherein the active ingredient fruquintinib is contained in an amount of 5 wt % or less, belong to low dose pharmaceutical compositions. For such compositions, it is generally not possible to prepare a pharmaceutical preparation having good drug content uniformity by simply mixing the active ingredient with the auxiliary materials. In addition, since the fruquintinib bulk drug substance has a long fibrous appearance and is adhesive, it is easy to block the mesh hole when sifting, thereby affecting the uniformity of drug content.

Accordingly, the present invention also provides a method for preparing the above pharmaceutical composition of fruquintinib, which comprises the steps of: premixing the furoquininib bulk drug substance with a portion of the filler, sieving, and then adding the remaining auxiliary materials and mixing evenly to obtain the pharmaceutical composition of the present invention.

In some embodiments of the present invention, the method of preparing the above-described pharmaceutical composition of fruquintinib comprises the steps of: premixing furoquininib bulk drug with a portion of microcrystalline cellulose, sieving, and then adding the remaining auxiliary materials and mixing evenly to obtain the pharmaceutical composition of the present invention.

In some embodiments of the present invention, the method of preparing the above-described pharmaceutical composition of fruquintinib comprises the steps of: pre-mixing furoquininib bulk drug with a portion of starch, sieving, and then adding the remaining auxiliary materials and mixing evenly to obtain the pharmaceutical composition of the present invention.

In some embodiments of the present invention, in the method of preparing the above pharmaceutical composition of fruquintinib, the fruquintinib bulk drug substance is previously subjected to a micronized pulverization treatment, and the particle size range (D90) is controlled to be less than about 35 μm, for example, the particle size range (D90) is controlled in the range of about 5˜30 μm.

In some embodiments of the present invention, in the method of preparing the above pharmaceutical composition of fruquintinib, the particle size range (D90) of the fruquintinib bulk drug substance is controlled to be less than about 15 μm, such as to control the particle size range (D90) to be in the range of about 5˜15 μm. In a particular embodiment of the invention, the fruquintinib bulk drug substance has a particle size (D90) of about 7.9 μm. In a particular embodiment of the invention, the fruquintinib bulk drug substance has a particle size (D90) of 10.4 μm.

In some embodiments of the present invention, in the method of preparing the above pharmaceutical composition of fruquintinib, the fruquintinib bulk drug substance has a particle size (D90) of about 18.5 μm. In one embodiment of the invention, the fruquintinib bulk drug substance has a particle size (D90) of about 35 μm.

The pharmaceutical composition containing fruquintinib provided by the present invention can be formulated into various dosage forms suitable for oral administration, such as tablets or capsules.

In some embodiments of the present invention, the pharmaceutical composition containing fruquintinib of the present invention is filled into capsules to prepare capsule formulation.

The present invention also provides the use of the above pharmaceutical composition containing fruquintinib for the preparation of a medicament for treating cancer. In one embodiment of the present invention, the cancer is selected from the group consisting of colorectal cancer, non-small cell lung cancer, and gastric cancer.

The present invention also provides a method of treating cancer with the above-described pharmaceutical composition of fruquintinib in combination with one or more other anti-tumor agents, wherein said pharmaceutical composition of fruquintinib is administered simultaneously or sequentially with other anti-tumor agents, for example, administered before or after other anti-tumor agents. In one embodiment of the present invention, the additional anti-tumor agent is docetaxel or gefitinib.

### DRAWINGS

FIG. 1 shows the dissolution profiles of capsules comprising compositions of the invention in 0.1M hydrochloric acid.

### EXAMPLES

The following non-limiting examples are provided to further illustrate the invention.

In all examples, the dissolution was tested by “the first method of dissolution (Basket Method)” according to Chinese pharmacopoeia, using 0.1 mol/L hydrochloric acid at 37° C. as the dissolution medium, setting the rotation speed at 100 rpm, sampling at appropriate time intervals and replenishing with the same volume of dissolution medium. After filtering with a 0.45 μm filter, the dissolution rate was calculated on the basis of the content measured by HPLC. The content uniformity was determined by sampling by a suitable method, measuring the content by HPLC method and calculating the RSD. The angle of repose is calculated from manual measurements.

### Example 1. Preparation of Fruquintinib Capsule

| Ingredients | Parts by weight | |
|---|---|---|
| Fruquintinib | 1 | part |
| Starch | 1040 | parts |

Weigh appropriate amount of fruquintinib (particle size D90=18.5 μm), pre-mix it with starch in amount of 20 times that of fruquintinib, and then sieve together. Sticking to the screen occurred. Then the remaining amount of starch is added and the mixture is mixed evenly with a V-type mixer. The mixture has good flowability, and the angle of repose is 41°. The content RSD of the mixture is 0.6%, which conforms to the control criterion of mixing uniformity (RSD≤5%). The mixture is filled in size #1 capsule, and sticking occurred during handling.

### Example 2. Preparation of Fruquintinib Capsule

| Ingredients | Parts by weight |
|---|---|
| Fruquintinib | 1 part |
| Microcrystalline cellulose | 52 parts |

Weigh appropriate amount of fruquintinib (particle size D90=18.5 μm), pre-mix it with microcrystalline cellulose in amount of 20 times that of fruquintinib, and then sieve together. Sticking to the screen occurred. Then the remaining amount of microcrystalline cellulose is added and the mixture is mixed evenly with a V-type mixer. The mixture has poor flowability, and the angle of repose is 52°. The content RSD of the mixture is 0.3%, which conforms to the control standard of mixing uniformity (RSD≤5%). The mixture is filled in size #1 capsule, and sticking occurred during handling.

### Example 3. Preparation of Fruquintinib Capsule

| Ingredients | Parts by weight | |
|---|---|---|
| Fruquintinib | 1 | part |
| Starch | 508.6 | parts |
| Microcrystalline cellulose | 520 | parts |
| Talc | 10.4 | parts |

Weigh appropriate amount of fruquintinib (particle size D90=18.5 μm), microcrystalline cellulose in amount of 10 times that of fruquintinib and starch in amount of 10 times that of fruquintinib, pre-mix and sieve together. No sticking occurs. Then add the remaining auxiliary materials according to the proportion, and the mixture is mixed evenly with a V-type mixer. The mixture has good flowability, and the angle of repose is 38°. The content RSD of the mixture is 0.6%, which conforms to the control criterion of mixing uniformity (RSD≤5%). The mixture is filled in size #1 capsule, and no sticking occurred during filling. The dissolution rate of the capsule in 0.1M hydrochloric acid at 30 minutes is 96.2%, which conforms to the dissolution specification (≥80%).

### Example 4. Preparation of Fruquintinib Capsule

| Ingredients | Parts by weight | |
|---|---|---|
| Fruquintinib | 1 | part |
| Microcrystalline cellulose PH 101 | 520 | parts |
| Starch | 520 | parts |

Weigh appropriate amount of fruquintinib (particle size D90=18.5 μm), microcrystalline cellulose in amount of 10 times that of fruquintinib and starch in amount of 10 times that of fruquintinib, pre-mix and sieve together. No sticking occurs. Then add the remaining amount of microcrystalline cellulose and starch according to the proportion, and the mixture is mixed evenly with a V-type mixer. The mixture has good flowability, and the angle of repose is 40°. The content RSD of the mixture is 0.6%, which conforms to the control criterion of mixing uniformity (RSD≤5%). The mixture is filled in size #1 capsule, and partial sticking occurred during filling. The dissolution rate of the capsule in 0.1M hydrochloric acid at 30 minutes is 82.1%, which is slightly slower, but conforms to the dissolution specification (≥80%).

### Example 5. Preparation of Fruquintinib Capsule

| Ingredients | Parts by weight | |
|---|---|---|
| Fruquintinib | 1 | part |
| Starch | 520 | parts |
| Microcrystalline cellulose | 520 | parts |
| Magnesium stearate | 2.6 | parts |

Weigh appropriate amount of fruquintinib (particle size D90=18.5 μm), microcrystalline cellulose in amount of 10 times that of fruquintinib and starch in amount of 10 times that of fruquintinib, pre-mix and sieve together. No sticking occurs. Then add the remaining auxiliary materials according to the proportion, and the mixture is mixed evenly with a V-type mixer. The mixture has good flowability, and the angle of repose is 38°. The content RSD of the mixture is 2.5%, which conforms to the control criterion of mixing uniformity (RSD≤5%). The mixture is filled in size #1 capsule, and no sticking occurred during filling. The dissolution rate of the capsule in 0.1M hydrochloric acid at 30 minutes is 94.0%, which conforms to the dissolution specification (≥80%).

### Example 6. Preparation of Fruquintinib Capsule

| Ingredients | Parts by weight | |
|---|---|---|
| Fruquintinib | 1 | part |
| Starch | 26 | parts |
| Microcrystalline cellulose | 26 | parts |
| Magnesium stearate | 0.13 | part |

Weigh appropriate amount of fruquintinib (particle size D90=18.5 μm), microcrystalline cellulose in amount of 10 times that of fruquintinib and starch in amount of 10 times that of fruquintinib, pre-mix and sieve together. No sticking occurs. Then add the remaining auxiliary materials according to the proportion, and the mixture is mixed evenly with a V-type mixer. The mixture has relatively good flowability, and the angle of repose is 46°. The content RSD of the mixture is 0.2%, which conforms to the control criterion of mixing uniformity (RSD≤5%). The mixture is filled in size #1 capsule, and no sticking occurred during filling. The dissolution rate of the capsule in 0.1M hydrochloric acid at 30 minutes is 88.2%, which is slightly slower than other formulations, but conforms to the dissolution specification (≥80%).

### Example 7. Preparation of Fruquintinib Capsule

| Ingredients | Parts by weight | |
|---|---|---|
| Fruquintinib | 1 | part |
| Microcrystalline cellulose PH 101 | 26 | parts |
| Starch | 26 | parts |
| Talc | 0.52 | parts |

Weigh appropriate amount of fruquintinib (particle size D90=18.5 μm), microcrystalline cellulose in amount of 10 times that of fruquintinib and starch in amount of 10 times that of fruquintinib, pre-mix and sieve together. Then add the remaining auxiliary materials according to the proportion, and the mixture is mixed evenly with a V-type mixer. The mixture has relatively good flowability, and the angle of repose is 46°. The content RSD of the mixture is 0.2%, which conforms to the control crierion of mixing uniformity (RSD≤5%). The mixture is filled in size #1 capsule, and no sticking occurred during filling. The dissolution rate of the capsule in 0.1M hydrochloric acid at 30 minutes is 91.1%, which conforms to the dissolution specification (≥80%).

### Example 8. Preparation of Capsules with Bulk Drug Substance of Different Particle Size

The ingredients of the formulations are the same as Example 3 as follows:

| Ingredients | Parts by weight | |
|---|---|---|
| Fruquintinib | 1 | part |
| Microcrystalline cellulose PH 101 | 520 | parts |
| Starch | 508.6 | parts |
| Talc | 10.4 | parts |

The particle size of the fruquintinib bulk drug substance used is as follows:

| No. | Particle size D90 (μm) |
|---|---|
| Example 8-1 | 7.9 |
| Example 8-2 | 35.0 |
| Example 8-3 | 10.4 |
| Example 3 | 18.5 |

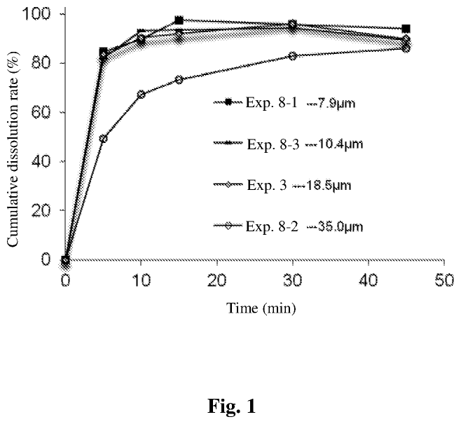

The preparation process of the capsule is the same as in Example 3. The dissolution profiles of the prepared capsules in 0.1M hydrochloric acid are compared and the results are shown in the following table and in FIG. 1.

RESULTS: The dissolution of the capsules prepared with the bulk drug substance having a particle size D90 of 35 μm in 0.1M hydrochloric acid is slightly slower than the other capsules, but all capsules meet the dissolution specification (≥80%) at 30 minutes. It can be seen that the dissolution will become slower as the particle size increases. It is recommended that the particle size D90 could be controlled not more than 35 μm.

| Particle size | | | | | | |
|---|---|---|---|---|---|---|
| of the bulk | | Average dissolution (%) n = 6 | | | | |
| drug | | Time (min) | | | | |
| d (0.9) | Example | 5 | 10 | 15 | 30 | 45 |
| 7.9 μm | 8-1 | 84.6 | 89.8 | 97.6 | 95.9 | 94.1 |
| 10.4 μm | 8-3 | 82.0 | 93.3 | 93.4 | 94.5 | 89.6 |
| 18.5 μm | 3 | 83.5 | 90.4 | 92.1 | 96.2 | 90.0 |
| 35.0 μm | 8-2 | 49.3 | 67.4 | 73.4 | 83.0 | 86.1 |

1. A pharmaceutical composition comprising fruquintinib, wherein the composition comprises the active ingredient fruquintinib and fillers and optionally a lubricant.

2. The composition according to claim 1, wherein the filler is selected from the group consisting of starch, microcrystalline cellulose and a combination thereof.

3. The composition according to claim 1, wherein the filler is a combination of starch and microcrystalline cellulose.

4. The composition according to claim 3, wherein the weight ratio of microcrystalline cellulose to starch is (0.9-1.1):1, preferably about 1:1.

5. The composition according to any one of claims 1-4, wherein the lubricant is selected from the group consisting of magnesium stearate, talc and a combination thereof.

6. The composition according to any one of claims 1-5, wherein the weight ratio of the lubricant to the filler is from about 1:50 to 1:1000, preferably from about 1:100 to 1:500.

7. The composition according to any one of claims 1-4, which is free of a lubricant.

8. The composition according to any one of claims 1-7, wherein the fruquintinib bulk drug substance has a particle size D90 of less than about 35 μm, such as in the range of about 5˜30 μm, such as in the range of about 5˜15 μm, for example, about 7.9 μm, about 10.4 μm, and about 18.5 μm.

9. The composition according to any one of claims 1-8, wherein the content of fruquintinib is in the range of about 0.001 wt % to 5 wt %, such as 0.01 to 5% by weight, such as 0.05 wt %, 1 wt %, 2 wt %, 3 wt %, based on the total weight of the pharmaceutical composition.

10. The composition according to any one of claims 1-9, in the form of a tablet or capsule.

11. A method of preparing a composition according to any one of claims 1-9, comprising the steps of: premixing the fruquintinib bulk drug substance with a portion of the filler, sieving, then adding the remaining auxiliary materials and mixing evenly.

12. Use of a composition according to any one of claims 1-9 in the manufacture of a medicament for the treatment of cancer.

13. The use according to claim 12, wherein the cancer is selected from the group consisting of colorectal cancer, non-small cell lung cancer, and gastric cancer.

14. The use according to claim 12 or 13, wherein the medicament is administered simultaneously or sequentially with other anti-tumor agents.

15. The use according to claim 14, wherein the other anti-tumor agent is docetaxel or gefitinib.
